SEQUENCE LISTING (42 sequences)

SEQ ID NO: 1 - DNA, 30 bp, Artificial Sequence
  Synthetic Construct
acgcgaattcatgaaaaaggtatgtgttat

SEQ ID NO: 2 - DNA, 47 bp, Artificial Sequence
  Synthetic Construct
gcgtctgcaggagctcctgtctctagaatttgataatggggattctt

SEQ ID NO: 3 - DNA, 30 bp, Artificial Sequence
  Synthetic Construct
acgcgagctctatagaattggtaaggatat

SEQ ID NO: 4 - DNA, 30 bp, Artificial Sequence
  Synthetic Construct
gcgtgagctcattgaacctccttaataact

SEQ ID NO: 5 - DNA, 30 bp, Artificial Sequence
  Synthetic Construct
agcttctagactcaagattaacgagtgcta

SEQ ID NO: 6 - DNA, 30 bp, Artificial Sequence
  Synthetic Construct
tagctctagattagtacattccgcccattc

SEQ ID NO: 7 - DNA, 26 bp, Artificial Sequence
  Synthetic Construct
agcgccatggattttaatttaacaag

SEQ ID NO: 8 - DNA, 30 bp, Artificial Sequence
  Synthetic Construct
agtcggtacccctccttaaattatctaaaa

SEQ ID NO: 9 - DNA, 30 bp, Artificial Sequence
  Synthetic Construct
atacggatccgagattagtacggtaatgtt

SEQ ID NO: 10 - DNA, 30 bp, Artificial Sequence
  Synthetic Construct
gtacctgcagcttacctcctatctattttt

SEQ ID NO: 11 - DNA, 72 bp, Artificial Sequence
  Synthetic Construct
gtgaaaccagtaacgttatacgatgtcgcagagtatgccggtgtctcttagattgcagcattacacgtcttg

SEQ ID NO: 12 - DNA, 70 bp, Artificial Sequence
  Synthetic Construct
tcactgcccgctttccagtcgggaaacctgtcgtgccagctgcattaatgcacttaacggctgacatggg

SEQ ID NO: 13 - DNA, 72 bp, Artificial Sequence
  Synthetic Construct
atgaaactcgccgtttatagcacaaaacagtacgacaagaagtacctgcagattgcagcattacacgtcttg

SEQ ID NO: 14 - DNA, 71 bp, Artificial Sequence
  Synthetic Construct
ttaaaccagttcgttcgggcaggtttcgcctttttccagattgcttaagtcacttaacggctgacatggga

SEQ ID NO: 15 - DNA, 71 bp, Artificial Sequence; identical to SEQ ID NO: 14
  Synthetic Construct

SEQ ID NO: 16 - DNA, 47 bp, Artificial Sequence; identical to SEQ ID NO: 2
  Synthetic Construct

SEQ ID NO: 17 - DNA, 30 bp, Artificial Sequence
  Synthetic Construct
acgctctagatataaggcatcaaagtgtgt

SEQ ID NO: 18 - DNA, 30 bp, Artificial Sequence
  Synthetic Construct
gcgtgagctccatgaagctaatataatgaa

SEQ ID NO: 19 - DNA, 30 bp, Artificial Sequence; identical to SEQ ID NO: 3
  Synthetic Construct

SEQ ID NO: 20 - DNA, 30 bp, Artificial Sequence; identical to SEQ ID NO: 4
  Synthetic Construct

SEQ ID NO: 21 - DNA, 30 bp, Artificial Sequence
  Synthetic Construct
atacgagctctacggcgagcaatggatgaa

SEQ ID NO: 22 - DNA, 30 bp, Artificial Sequence
  Synthetic Construct
gtacgagctcgattaattcaaccgttcaat

SEQ ID NO: 23 - DNA, 39 bp, Artificial Sequence
  Synthetic Construct
agtcgagctcaggaaacagatgactgacgttgtcatcgt

SEQ ID NO: 24 - DNA, 30 bp, Artificial Sequence
  Synthetic Construct
atgcgagctcttatttgcgctcgactgcca

SEQ ID NO: 25 - DNA, 30 bp, Artificial Sequence
  Synthetic Construct
agcttctagagatgggttacctgacatata

SEQ ID NO: 26 - DNA, 30 bp, Artificial Sequence
  Synthetic Construct
agtctctagattatgactcaaacgcttcag

SEQ ID NO: 27 - DNA, 30 bp, Artificial Sequence
  Synthetic Construct
agcttctagaactgctccttggacagcgcc

SEQ ID NO: 28 - DNA, 30 bp, Artificial Sequence
  Synthetic Construct
agtctctagaggcaggcaggatcagaacca

SEQ ID NO: 29 - DNA, 30 bp, Artificial Sequence
  Synthetic Construct
agcttctagaactgttccttggacagcgcc

SEQ ID NO: 30 - DNA, 30 bp, Artificial Sequence; identical to SEQ ID NO: 28
  Synthetic Construct

SEQ ID NO: 31 - DNA, 26 bp, Artificial Sequence
  Synthetic Construct
agcgccatggattttaatttaacaag

SEQ ID NO: 32 - DNA, 30 bp, Artificial Sequence; identical to SEQ ID NO: 8
  Synthetic Construct

SEQ ID NO: 33 - DNA, 30 bp, Artificial Sequence
  Synthetic Construct
acgcggatccgtagtttgcatgaaatttcg

SEQ ID NO: 34 - DNA, 41 bp, Artificial Sequence
  Synthetic Construct
agtcctgcagctatcgagctctataatggctacgcccaaac

SEQ ID NO: 35 - DNA, 30 bp, Artificial Sequence
  Synthetic Construct
actcgagctcaaaagccgagattagtacgg

SEQ ID NO: 36 - DNA, 30 bp, Artificial Sequence
  Synthetic Construct
gcgtctgcagcctatctatttttgaagcct

SEQ ID NO: 37 - DNA, 30 bp, Artificial Sequence
  Synthetic Construct
atgcgaattcatgagtaagcgtaaagtcgc

SEQ ID NO: 38 - DNA, 48 bp, Artificial Sequence
  Synthetic Construct
tatcctgcaggagctctctagagctagcttaccgttcatgccgcttct

SEQ ID NO: 39 - DNA, 30 bp, Artificial Sequence
  Synthetic Construct
atacgctagcatgaactggccgcaggttat

SEQ ID NO: 40 - DNA, 30 bp, Artificial Sequence
  Synthetic Construct
tatcgagctcgccaggccttatgactcata

SEQ ID NO: 41 - DNA, 30 bp, Artificial Sequence
  Synthetic Construct
atacgagctctgcatcactgccctgctctt

SEQ ID NO: 42 - DNA, 30 bp, Artificial Sequence
  Synthetic Construct
tgtcgagctccgctatcgggtgtttttatt

(19) United States
(12) Patent Application Publication
(10) Pub. No.: US 2011/0020888 A1
(43) Pub. Date: Jan. 27, 2011

(54) METHOD FOR PREPARING BUTANOL THROUGH BUTYRYL-CoA AS AN INTERMEDIATE USING BACTERIA

(75) Inventors: Eleftherios Terry Papoutsakis, Newark, DE (US); Sang Yup Lee, Daejeon (KR); Jin Hwan Park, Gyeonggi-do (KR)

Correspondence Address: MOORE & VAN ALLEN PLLC, P.O. BOX 13706, Research Triangle Park, NC, 27709, US

(73) Assignee: BIOFUELCHEM CO., LTD., Daejeon (KR)

(21) Appl. No.: 12/518,553

(22) PCT Filed: Dec. 14, 2007
(86) PCT No.: PCT/KR07/06524

§ 371 Date: Oct. 13, 2010

Related U.S. Application Data
(60) Provisional application No. 60/875,145, filed on Dec. 15, 2006.
(60) Provisional application No. 60/899,201, filed on Feb. 2, 2007.

(51) Int. Cl. C12P 7/16

(52) U.S. Cl. 435/160

(57) ABSTRACT

The present invention relates to a method for producing butanol using a bacterium capable of biosynthesizing butanol from butyryl-CoA as an intermediate. More particularly, a method for producing butanol, the method comprising generating bytyryl-CoA in a bacterium which contains a gene coding for AdhE (an enzyme responsible for the conversion of butyryl-CoA to butanol) using various methods, and converting the butyryl-CoA into butanol.

### TECHNICAL FIELD

The present invention relates to a method for producing butanol in bacteria capable of biosynthesizing butanol using butyryl-CoA as an intermediate.

### BACKGROUND ART

With the great increase in oil prices and growing concern about global warming and greenhouse gases, biofuels have recently gained increasing attention with respect to the production thereof using microorganisms. Particularly, biobutanol has an advantage over bioethanol in that it is more highly miscible with fossil fuels thanks to the low oxygen content thereof. Recently emerging as a substitute fuel for gasoline, biobutanol has rapidly increased in market size. The U.S. market for biobutanol amounts to 370 million gal per year, with a price of 3.75 $/gal. Butanol is superior to ethanol as a replacement for petroleum gasoline. With high energy density, low vapor pressure, a gasoline-like octane rating and low impurity content, it can be blended into existing gasoline at much higher proportions than ethanol without compromising performance, mileage, or organic pollution standards. The mass production of butanol by microorganisms can confer economic and environmental advantages of decreasing the import of crude oil and greenhouse gas emissions.

Butanol can be produced through anaerobic ABE (acetone-butanol-ethanol) fermentation by Clostridial strains (Jones, D. T. and Woods, D. R., Microbiol. Rev., 50:484, 1986; Rogers, P., Adv. Appl. Microbiol., 31:1, 1986; Lesnik, E. A. et al., Necleic Acids Research, 29: 3583, 2001). This biological method was the main technology for the production of butanol and acetone for more than 40 years, until the 1950s. Clostridial strains are difficult to improve further because of complicated growth conditions thereof and the insufficient provision of molecular biology tools and omics technology therefor.

Thus, it is suggested that microorganisms such as E. coli that can grow rapidly under typical conditions and be manipulated using various omics technologies be developed as butanol-producing strains. Particularly, E. coli species, to which little metabolic engineering and omics technology have been applied for the development of butanol-producing strains, have vast potential for development into butanol-producing strains.

Clostridium acetobutylicum produces butanol through the butanol biosynthesis pathway shown in FIG. 1 (Jones, D. T. and Woods, D. R., Microbiol. Rev., 50:484, 1986; Desai, R. P. et al., J. Biotechnol., 71:191, 1999). In wild-type E. coli, ethanol is synthesized via a similar pathway in which adhE (coding for the AdhE enzyme responsible for the production of ethanol from acetyl-CoA through acetaldehyde) inducible under anaerobic conditions plays a critical role. E. coli may contain some of the genes necessary for the biosynthesis of butyryl-CoA and butanol, but the expression level thereof is too low to effectively catalyze the corresponding enzyme reactions, unlike it's the corresponding genes in Clostridia.

Meanwhile, recombinant bacteria capable of producing butanol, into which a butanol biosynthesis pathway is introduced, and butanol production using the same have been disclosed (US 2007/0259410 A1; US 2007/0259411 A1), but the production efficiency is modest.

Therefore, the present inventors have made extensive efforts to develop a nivel method for producing butanol using bacteria (particularly, E. coli), produced butyryl-CoA as an intermediate in bacteria containing a gene coding for an enzyme (AdhE) responsible for the conversion of butyryl-CoA to butanol using various methods, and confirmed that the produced butyryl-CoA is converted to butanol by AdhE.

### SUMMARY OF THE INVENTION

Accordingly, it is an object of the present invention to provide various methods for generating butyryl-CoA which is an important intermediate in biosynthesis pathway of butanol and the like.

It is another object of the present invention to provide a method for producing butanol using bacteria capable of biosynthesizing butanol from butyryl-CoA as an intermediate.

In order to accomplish the above objects, the present invention provides a method for producing butanol, the method comprising: culturing a recombinant bacterium containing a gene coding for an enzyme (AdhE) responsible for the conversion of butyryl-CoA to butanol, and into which a gene coding for CoAT (acetyl-CoA: butyryl-CoA transferase) is introduced, in a butyrate or acetoacetate-containing culture medium to produce butanol; and recovering butanol from the culture broth.

The present invention also provides a method for generating butyryl-CoA, the method comprising: culturing a recombinant bacterium into which a gene coding for CoAT (acetyl-CoA: butyryl-CoA transferase) is introduced in a butyrate or acetoacetate-containing culture medium.

In addition, the present invention provides a method for generating butyryl-CoA, the method comprising: culturing a bacterium containing a gene coding for AtoDA (acetyl-CoA: acetoacetyl-CoA transferase) in a butyrate-containing culture medium.

Further, the present invention provides a method for producing butanol, the method comprising: culturing a bacterium containing a gene coding for AtoDA and a gene coding for AdhE in a butyrate-containing culture medium to produce butanol; and recovering butanol from the culture broth.

Moreover, the present invention provides a method for generating butyryl-CoA, the method comprising: culturing a bacterium containing genes coding for AtoDA (acetyl-CoA: acetoacetyl-CoA transferase), FadB or PaaH (3-hydroxyacyl-CoA dehydrogenase), PaaFG (enoyl-CoA hydratase) and FadE (acyl-CoA dehydrogenase) in a butyrate- or acetoacetate-containing culture medium.

Furthermore, the present invention provides a method for producing butanol, the method comprising: culturing a bacterium containing genes coding for AtoDA, FadB or PaaH, PaaFG and FadE together with a gene coding for AdhE in a butyrate- or acetoacetate-containing culture medium to produce butanol; and recovering butanol from the culture broth.

The present invention also provides a recombinant bacterium having butanol producing ability, into which genes coding for thiolase (THL), 3-hydroxybutyryl-CoA dehydrogenase (BHBD), crotonase (CRO) and functional BCD (butyryl-CoA dehydrogenase) are introduced, and a method for producing butanol, the method comprising: culturing the recombinant bacterium in a culture medium to produce butanol; and recovering butanol from the culture broth.

The present invention also provides a method for generating butyryl-CoA, the method comprising: culturing a recombinant bacterium into which genes coding for THL, BHBD, crotonase and functional BCD are introduced.

The present invention also provides a recombinant bacterium having butanol producing ability into which genes coding for THL, BHBD, crotonase, functional BCD, AAD, BDH and a chaperone protein are introduced and a lacI gene (coding for a lac operon repressor) and a gene coding for an enzyme involved in lactate biosynthesis are deleted, and a method for producing butanol, the method comprising: culturing the recombinant bacterium in a culture medium to produce butanol; and recovering butanol from the culture broth.

Other features and aspects of the present invention will be apparent from the following detailed description and the appended claims.

### BRIEF DESCRIPTION OF DRAWINGS

FIG. 1 is a schematic diagram showing a butanol biosynthesis pathway in Clostridium acetobutylicum.

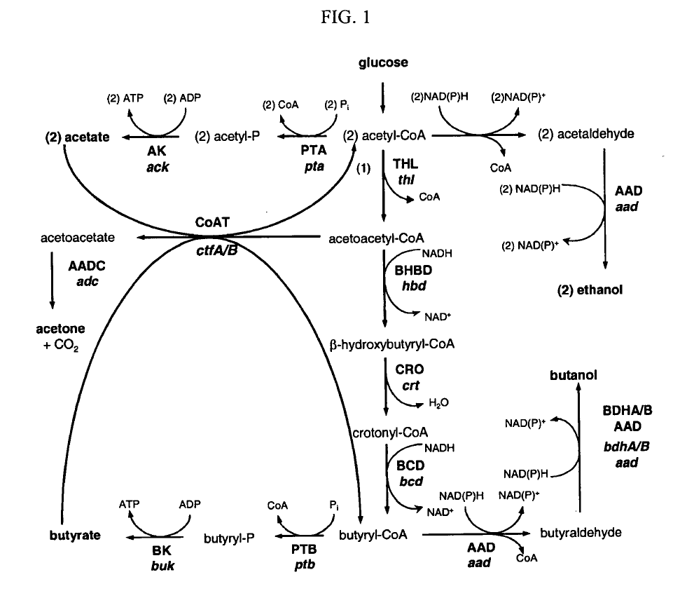

FIG. 2 is a schematic diagram showing a putative butanol biosynthesis pathway in the recombinant E. coli according to the present invention.

FIG. 3 is a schematic diagram showing a biosynthesis pathway that result in producing butanol via butyryl-CoA in an ato system and/or fad system.

FIG. 4 shows a pathway for conversion of acetyl-CoA to butyryl-CoA in Clostridium acetobutylicum.

FIG. 5 shows a construction process and a genetic map of a pKKhbdthiL vector.

FIG. 6 shows a construction process and a genetic map of a pTrc184bcdcrt vector.

FIG. 7 shows a construction process and a genetic map of pKKhbdadhEthiL (pKKHAT) vector.

FIG. 8 shows a construction process and a genetic map of pKKhbdadhEatoB (pKKHAA) vector.

FIG. 9 shows a construction process and a genetic map of pKKhbdadhEphaA (pKKHAP) vector.

FIG. 10 shows a construction process and a genetic map of pKKhbdydbMadhEphaA (pKKHYAP) vector.

FIG. 11 shows a construction process and a genetic map of pKKhbdbcdPA01adhEphaA (pKKHPAP) vector.

FIG. 12 shows a construction process and a genetic map of pKKhbdbcdKT2440adhEphaA (pKKHKAP) vector.

FIG. 13 shows a construction process and a genetic map of pTrc184bcdbdhABcrt (pTrc184BBC) vector.

FIG. 14 is shows a butanol biosynthesis pathway in the case where a part of genes derived from C. acetobutylicum involved in a butanol biosynthesis pathway, was substituted by genes derived from E. coli.

FIG. 15 shows a construction process and a genetic map of pKKmhpFpaaFGHatoB (pKKMPA) vector.

### DETAILED DESCRIPTION OF THE INVENTION, AND PREFERRED EMBODIMENTS

In the present invention, an examination was made of whether the recombinant E. coli [ATCC 11303(pACT)], which harbors genes coding for thiolase (THL; gene: thl or thiL); acetyl-CoA: butyryl-CoA CoA-transferase (CoAT; gene: ctfA and ctfB); and acetoacetate decarboxylase (AADC; gene: adc), derived from Clostridium acetobutylicum, can produce butanol from butyryl-CoA by means of its endogenous enzyme (AdhE, expressed under anaerobic conditions). This recombinant E. coli [ATCC 11303(pACT)] was constructed so as to produce acetone from acetyl-CoA through acetoacetyl-CoA (Bermejo, L. L. et al., Appl. Environ. Microbiol., 64:1079, 1998).

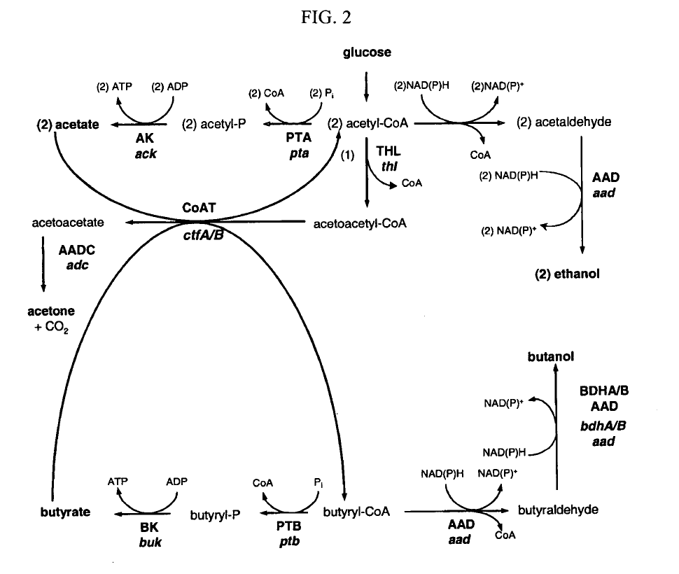

It is expected that, when CoA residue of acetoacetyl-CoA is replaced using the CoAT enzyme of Clostridium acetobutylicum (responsible for the conversion of butyric acid (BA) or acetic acid into butyryl-CoA or acetyl-CoA), which is expressed by the recombinant E. coli, butyryl-CoA can be produced (FIG. 2). Also, it is expected that the AdhE enzyme of the recombinant E. coli (ATCC 11303(pACT)), which is expressed under anaerobic conditions and is responsible for the conversion of acetyl-CoA into ethanol, catalyzes the conversion of butyryl-CoA to butanol, to produce butanol (FIG. 2).

To confirm the above prediction, the recombinant E. coli was cultured in a butyrate-containing medium, and as a result, it could be seen that butyrate was converted through butyryl-CoA to butanol, suggesting that it is due to the CoAT enzyme encoded by the ctfA and ctfB genes introduced into the recombinant in concert with the AdhE enzyme expressed under an anaerobic condition.

In the following examples, the recombinant E. coli was verified to produce butanol when it was cultured in a medium containing butyrate and/or acetoacetate.

Therefore, the present invention, in one aspect, relates to a method for producing butanol, the method comprising: culturing a recombinant bacterium containing a gene coding for an enzyme (AdhE) responsible for the conversion of butyryl-CoA to butanol, and into which a gene coding for CoAT (acetyl-CoA: butyryl-CoA transferase) is introduced, in a butyrate or acetoacetate-containing culture medium to produce butanol; and recovering butanol from the culture broth.

The present invention also relates to a method for generating butyryl-CoA, the method comprising: culturing a recombinant bacterium into which a gene coding for CoAT (acetyl-CoA: butyryl-CoA transferase) is introduced in a butyrate or acetoacetate-containing culture medium.

In the present invention, preferably, genes coding for thiolase (THL) and acetoacetate decarboxylase (AADC) are additionally introduced into the recombinant bacterium.

Preferably, the CoAT (acetyl-CoA: butyryl-CoA transferase) useful in the present invention may be encoded by ctfA and ctfB genes derived from Clostridium, but the present invention is not limited thereto. Also, the THL expressed in the recombinant microorganism of the present invention is preferably encoded by thl or thiL derived from Clostridium sp., phaA derived from Ralstonia sp., or atoB derived from E. coli, but is not limited thereto. Preferably, the AADC expressed in the recombinant microorganism of the present invention is encoded by the adc gene derived from Clostridium sp., but is not limited thereto. As long as it is expressed as an enzyme having the same activity in the host bacterium, any exogenous gene can be used in the present invention without limitation.

In the present invention, the host bacterium is preferably E. coli. However, as long as it harbors a gene coding for AdhE, it is not limited thereto.

In an example of the present invention, butanol was detected when a wild-type E. coli with no pACT introduced thereinto was cultured in a medium containing butyrate and/or acetoacetate.

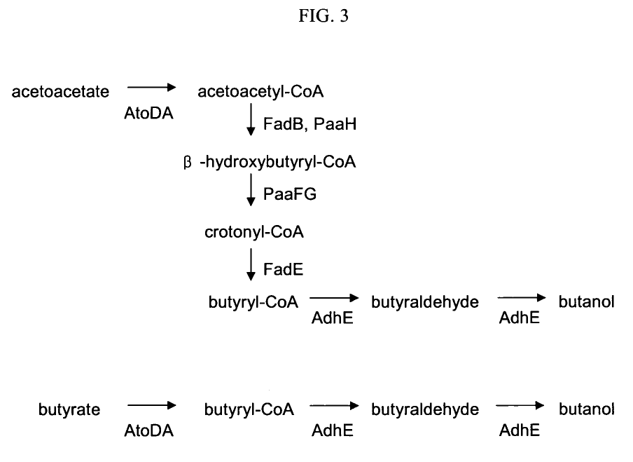

The production of butanol by the wild-type E. coli cultured in a butyrate-containing medium is believed to result from the conversion of butyrate into butyryl-CoA by AtoDA of the ato system (Lioliou and Kyriakidis, Microbial Cell Factories, 3:8, 2004) and then to butanol by E. coli AdhE enzyme (FIG. 3). AtoDA, wherein AtoD represents an acetyl-CoA: acetoacetyl-CoA transferase α subunit and AtoA represents an acetyl-CoA: acetoacetyl-CoA transferase β subunit, is an enzyme responsible for the following reaction:

aa-CoA+acetate (or butyrate)←→aa+acetyl (butyryl)-CoA

Therefore, the present invention, in another aspect, relates to a method for producing butanol, the method comprising: culturing a bacterium containing a gene coding for AtoDA (acetyl-CoA: acetoacetyl-CoA transferase) and a gene coding for AdhE responsible for the conversion of butyryl-CoA to butanol in a butyrate-containing culture medium to produce butanol; and recovering butanol from the culture broth.

The present invention also relates to a method for generating butyryl-CoA, the method comprising: culturing a bacterium containing a gene coding for AtoDA (acetyl-CoA: acetoacetyl-CoA transferase) in a butyrate-containing culture medium.

In the present invention, the bacterium containing a gene coding for AtoDA and/or a gene coding for AdhE is preferably E. coli, but it is not limited thereto as long as it harbors the above genes.

In addition, the production of butanol by the wild-type E. coli cultured in an acetoacetate-containing medium is assumed to result from the conversion of acetoacetate into acetoacetyl-CoA by AtoDA of the ato system, then into butyryl-CoA by FadB (or PaaH), PaaFG and FadE of the fad system (Park and Lee, Biotechnol. Bioeng., 86:681, 2004), and finally into butanol by E. coli AdhE enzyme (FIG. 3).

FadB is known to have four functions: 3-hydroxyacyl-CoA dehydrogenase; 3-hydroxybutyryl-CoA epimerase; delta(3)-cis-delta(2)-trans-enoyl-CoA isomerase; and enoyl-CoA hydratase, and is involved, together with FadA, in the following reaction:

acyl-CoA+acetyl-CoA←→CoA+3-oxoacyl-CoA

FadB (or PaaH) functions to convert acetoacetyl-CoA to β-hydroxybutyryl-CoA. PaaFG is enoyl-CoA hydratase responsible for the conversion of β-hydroxybutyryl-CoA to crotonyl-CoA. FadE is acyl-CoA dehydrogenase, involved in the following reaction, for converting crotonyl-CoA to butyryl-CoA:

Butanoyl-CoA+FAD←→FADH2+Crotonoyl-CoA

Therefore, the present invention, in still another aspect, relates to a method for producing butanol, the method comprising: culturing a bacterium containing genes coding for AtoDA, FadB or PaaH, PaaFG and FadE together with a gene coding for AdhE responsible for the conversion of butyryl-CoA to butanol in a butyrate- or acetoacetate-containing culture medium to produce butanol; and recovering butanol from the culture broth.

The present invention also relates to a method for generating butyryl-CoA, the method comprising: culturing a bacterium containing genes coding for AtoDA (acetyl-CoA: acetoacetyl-CoA transferase), FadB or PaaH (3-hydroxyacyl-CoA dehydrogenase), PaaFG (enoyl-CoA hydratase) and FadE (acyl-CoA dehydrogenase) in a butyrate- or acetoacetate-containing culture medium.

In the present invention, E. coli is preferable for the bacterium that harbors a gene coding for AtoDA, a gene coding for FadB or PaaH, a gene coding for PaaFG and a gene coding for FadE, and/or a gene coding for AdhE. However, as long as it contains such a gene(s), any bacterium may be used in the present invention.

As described above, when a pathway for the conversion of acetyl-CoA to butyryl-CoA is introduced into a bacterium, such as E. coli containing a gene coding for an enzyme (AdhE) functioning to convert butyryl-CoA to butanol, the bacterium can produce butanol.

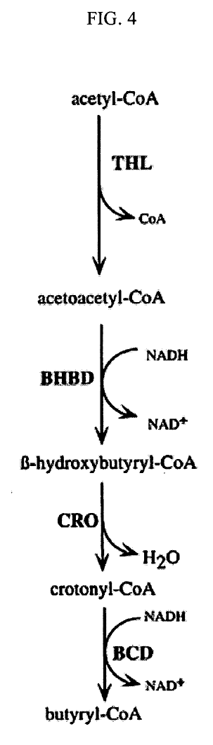

The pathway of Clostridium sp. is known to be a pathway for converting acetyl-CoA to butyryl-CoA (FIG. 4). In the pathway of FIG. 4, the gene thl from Clostridium sp. has already been identified to effectively express THL in E. coli (Bermejo, L. L. et al., Appl. Environ. Microbiol., 64:1079, 1998). In addition to thl, the gene thiL is known to encode THL derived from Clostridium sp. (Nolling, J. et al., J. Bacteriol., 183:4823, 2001). THL functions to convert acetyl-CoA into acetoacetyl-CoA. Also, in an example of the present invention, the introduction of phaA derived from Ralstonia sp., or atoB derived from E. coli was also found to give the bacterium THL activity in addition to thl or thiL derived from Clostridium sp., as detected with butanol production. Accordingly, phaA derived from Ralstonia sp., or atoB derived from E. coli can be used instead of thl or thiL. Further, as long as it is expressed to show THL activity in the host cells, any gene coding for THL, even if exogenous, can be used without limitations.

Also, Bennett et al. reported that among enzymes necessary for the production of butyryl-CoA from acetoacetyl-CoA, BHBD and CRO except for BCD are expressed in E. coli (Boynton, Z. L. et al., J. Bacteriol., 178:3015, 1996). According to the article, however, it is reported that E. coli has no BCD function because of the poor expression of BCD or its cofactors (electron transfer flavoproteins putatively coded by the Clostridium acetobutylicum genes (etfB and etfA)) therein, or no in vitro activity is observed because of the poor stability of BCD or its cofactors.

In the present invention, the low-level expression of butyryl-CoA dehydrogenase can be solved by introducing a gene (groESL) coding for a chaperon protein together with bcd derived from Clostridium acetobutylicum. In the example of the present invention, when the bcd derived from Clostridium acetobutylicum and the chaperone-encoding gene (groESL) are introduced into E. coli, the E. coli host cells were observed to increase in butanol production as demonstrated.

In an alternative, the low-level expression of butyryl-CoA dehydrogenase can be overcome by the introduction of bcd derived from Pseudomonas aeruginosa or Pseudomonas putida, or ydbM derived from Bacillus subtilis. Therefore, as long as it is expressed to show BCD activity in the host cells, a BCD gene, even though exogenous, can be used without limitations.

In an example of the present invention, it was confirmed that E. coli containing thiL, hbd, bcd, groESL and crt, derived from Clostridium sp. produces butanol from glucose through butyryl-CoA. In another example of the present invention, it was confirmed that E. coli containing bcd derived from Pseudomonas sp. or ydbM derived from Bacillus sp., instead of bcd and groESL derived from Clostridium sp., produces butanol from glucose through butyryl-CoA as an intermediate.

Therefore, the present invention, in still another aspect, relates to a recombinant bacterium having butanol producing ability, into which genes coding for thiolase (THL), 3-hydroxybutyryl-CoA dehydrogenase (BHBD), crotonase (CRO) and functional BCD (butyryl-CoA dehydrogenase) are introduced, and a method for producing butanol, the method comprising: culturing the recombinant bacterium in a culture medium to produce butanol; and recovering butanol from the culture broth.

The present invention also relates to a method for generating butyryl-CoA, the method comprising: culturing a recombinant bacterium into which genes coding for THL, BHBD, crotonase and functional BCD are introduced.

The intermediate butyryl-CoA thus produced is converted into butanol by the AdhE enzyme, which is encoded by the endogenous gene coding for AdhE in the bacterium. E. coli carries a gene coding for an enzyme (AdhE) for converting butyryl-CoA into butanol. In the case of a host cell which does not carry an AdhE-encoding gene, when genes coding for AAD (butyraldehyde dehydrogenase) and BDH (butanol dehydrogenase) are introduced, the host cell can produce butanol from butyryl-CoA. Even if a host cell harbors a gene coding for AdhE per se, when genes coding for AAD (butyraldehyde dehydrogenase) and BDH (butanol dehydrogenase) are introduced, the conversion of butyryl-CoA to butanol can be promoted by the expressed enzymes AdhE, AAD and BDH.

In accordance with an aspect of the present invention, preferably, the recombinant bacterium into which a gene coding for AAD (butyraldehyde dehydrogenase) and/or a gene coding for BDH (butanol dehydrogenase) are additionally introduced. The gene coding for AAD is preferably adhE derived from Clostridium sp. or mhpF derived from E. coli, but is not limited thereto. For example, ADD-encoding genes from microorganisms other than Clostridium sp. can be used as long as without limitation they are expressed to show the same AAD activity. Also, the gene coding for BDH is preferably bdhAB derived from Clostridium sp., but is not limited thereto. For example, genes from microorganisms other than Clostridium sp. may be used without limitation as long as they are expressed to show the same BDH activity.

In the present invention, the gene coding for THL may be preferably thl or thiL derived from Clostridium sp., phaA derived from Ralstonia sp., or atoB derived from E. coli. The genes coding for BHBD and crotonase may be preferably hbd and crt derived from Clostridium sp, respectively, but are not limited thereto. For example, any exogenous gene can be used without limitation as long as it is expressed to show BHBD (FadB or PaaH in E. coli) activity and crotonase (PaaFG in E. coli) activity in the host cells. In an example of the present invention, butanol was detected even in a culture in which paaH (coding for 3-hydroxyacyl-CoA dehydrogenase) and paaFG (coding for enoyl-CoA hydratase), derived from E. coli, were substituted for hbd and crt derived from Clostridium sp.

By the term “functional BCD”, as used herein, it is meant that a bcd gene introduced into a host cell, such as E. coli, is expressed to show BCD activity. Examples of the gene coding for functional BCD include bcd derived from Pseudomonas sp. and ydbM derived from Bacillus sp., but are not limited thereto. The bcd derived from Clostridium sp. may also be included in the functional BCD-encoding gene since it shows weak activity in E. coli an the like the BCD activity is amplified when it is introduced together with a gene (groESL) coding for a chaperone protein.

In accordance with an aspect of the present invention, preferably, the recombinant bacterium into which a gene coding for a chaperone protein is additionally introduced. The gene coding for a chaperone protein is preferably groESL.

In order to increase the expression of genes coding for enzymes responsible for butanol biosynthesis, the recombinant bacterium may preferably has lacI (coding for a lac operon repressor) deleted. Preferably, a gene coding for an enzyme involved in lactate biosynthesis is additionally deleted. The gene coding for the enzyme involved in lactate biosynthesis is preferably ldhA (coding for lactate dehydrogenase).

Finally, in the present invention a butanol-producing recombinant mutant E. coli was constructed by introducing genes coding for THL, BHBD, crotonase, functional BCD, AAD, BDH and a chaperone protein thereinto and deleting a lacI gene (coding for a lac operon repressor) and a gene coding for an enzyme responsible for lactate biosynthesis, thus confirming that butanol productivity is dramatically increased in said recombinant mutant E. coli.

The term “deletion”, as used herein in relation to a gene, means that the gene cannot be expressed or, if it is expressed, cannot lead to enzyme activity, due to the mutation, substitution, deletion or insertion of any number of nucleotides from a single base to an entire piece of the gene, resulting in the blockage of the biosynthesis pathway in which an enzyme encoded by gene is involved.

### EXAMPLES

A better understanding of the present invention may be obtained through the following examples which are set forth to illustrate, but are not to be construed as the limit of the present invention.

Although, in the following examples, E. coli W3110 was used as a host microorganism, it will be obvious to those skilled in the art that other E. coli strains, bacteria, yeasts and fungi can also be used as host cells by deleting target gene to be deleted and introducing genes involved in butanol biosynthesis, in order to produce butanol.

Further, although genes derived from a specific strain are exemplified as target genes to be introduced in the following examples, it is obvious to those skilled in the art that as long as they are expressed to show the same activity in the host cells, any genes may be employed without limitations.

Also, it should be noted that although only specific culture media and methods are exemplified in the following example, saccharified liquid, such as whey, CSL (corn steep liquor), etc, and the other media, and various culture methods, such as fed-batch culture, continuous culture, etc. (Lee et al., Bioprocess Biosyst. Eng., 26:63, 2003; Lee et al., Appl. Microbiol. Biotechnol., 58:663, 2002; Lee et al., Biotechnol. Lett., 25:111, 2003; Lee et al., Appl. Microbiol. Biotechnol., 54:23, 2000; Lee et al., Biotechnol. Bioeng., 72:41, 2001) also fall within the scope of the present invention.

### Example 1

### Production of Butanol by Butyrate Addition

An attempt was made to produce butanol by culturing recombinant E. coli [ATCC 11303(pACT)]. For this culture, the medium was used: LB medium, containing 10 g/L NaCl, 10 g/L Bacto tryptone and 5 g/L yeast extract+20 g/L glucose and 1 g/L NaHCO₃.

To 12 ml of the medium in a 15 ml culture tube, 50 μg/ml ampicillin was added and the recombinant E. coli [ATCC 1103(pACT)] was inoculated into the medium to culture for 1 hr in an aerobic chamber, followed by culturing for 2 hr in an anaerobic chamber.

Afterwards, the bacterium was cultured in the culture medium with 0.8 mM butyric acid in an amount of 50 μl, 100 μl, 200 μl or 300 μl every 2 hr added thereto. Before addition, the pH of butyric acid was adjusted to that of the culture medium.

After 24, 48, 72, 96, 140 and 164 hrs of incubation, the culture broth was analyzed for components thereof using HPLC (Table 1). In Table 1, ‘L-200-48’ means the culturing in a medium added with 200 μl of 0.8 mM butyric acid for 48 hr, and ‘C’ represents a control cultured in a medium without butyrate.

As a result, as shown in Table 1, none of ethanol, butanol, acetic acid or butyric acid was detected in the negative control, LB medium, while only acetate and ethanol were produced in the positive controls without adding butyric acid. With one exception, a low level of butanol was detected after 164 hr of culture, which might be derived from butyryl-CoA or might not. By contrast, when the recombinant E. coli [ATCC 11303(pACT)] was cultured with butyrate, ethanol was produced, which indicates that AdhE enzyme is expressed under an anaerobic condition and also the production of acetone indicates the expression of a CoAT enzyme, thus finally confirming that butanol was produced.

TABLE 1
HPLC analysis of supernatants from cultures of E. coli ATCC 11303 (pACT)
challenged with butyric acid (mM)

| Sample | Glucose | Acetate | Acetoin | Ethanol | Butyrate | Acetone | Butanol |
|---|---|---|---|---|---|---|---|
| LB | 127.282 | 0 | 0 | 0 | 0 | 0 | 0 |
| L-C-24 | 99.421 | 7.449 | 0 | 14.769 | 0 | 7.113 | 0 |
| L-C-48 | 98.327 | 7.425 | 0 | 14.52 | 0 | 6.142 | 0 |
| L-C-164 | 95.198 | 5.815 | 0 | 17.417 | 0 | 3.949 | 0.26 |
| L-50-24 | 99.214 | 9.38 | 0 | 14.609 | 5.57 | 6.018 | 0.33 |
| L-50-48 | 97.390 | 9.364 | 0 | 17.101 | 5.209 | 5.735 | 0.662 |
| L-100-24 | 97.690 | 10.487 | 0 | 12.761 | 11.641 | 5.652 | 0.878 |
| L-100-48 | 96.373 | 10.576 | 0 | 14.805 | 12.304 | 5.243 | 0.892 |
| L-200-24 | 98.303 | 11.444 | 0 | 10.211 | 24.713 | 4.333 | 0.978 |
| L-200-48 | 95.835 | 11.956 | 0 | 10.895 | 25.288 | 4.441 | 1.036 |
| L-200-72 | 95.588 | 11.824 | 0 | 12.887 | 25.443 | 4.441 | 1.064 |
| L-200N-72 | 94.990 | 11.78 | 0 | 13.12 | 31.75 | 4.63 | 1.16 |
| L-200N-96 | 90.985 | 9.338 | 0 | 13.526 | 30.192 | 3.893 | 0.891 |
| L-200N-140 | 90.604 | 9.717 | 0 | 10.314 | 27.805 | 2.078 | 1.256 |
| L-200N-164 | 95.132 | 10.095 | 0 | 10.946 | 29.089 | 1.759 | 1.21 |
| L-300-24 | 96.751 | 12.681 | 0 | 9.48 | 37.123 | 3.556 | 0.978 |
| L-300-48 | 93.274 | 13.288 | 0 | 12.962 | 36.676 | 4.247 | 1.358 |
| L-300-72 | 92.784 | 13.816 | 0 | 0.304 | 36.644 | 4.206 | 1.273 |
| L-300N-72 | 92.770 | 12.65 | 0 | 0 | 43.52 | 4.13 | 1.34 |
| L-300N-96 | 91.802 | 11.599 | 0 | 16.214 | 41.71 | 3.046 | 1.018 |
| L-300N-140 | 92.336 | 11.23 | 0 | 11.249 | 40.255 | 1.908 | 1.391 |
| L-300-164 | 92.971 | 11.404 | 0 | 3.34 | 42.652 | 2.259 | 1.306 |

N—new, additional challenge with butyric acid and brief exposure to oxygen
LB: growth medium with cell added

### Example 2

### Production of Butanol by Acetoacetate Addition

The culture procedure was the same as described in Example 1, with the exception that LB and M9 media containing acetoacetate and butyrate, were used. That is, the recombinant E. coli [ATCC 11303(pACT)] was cultured in LB (30 g/L glucose) and M9 (200 ml/L 5×M9 salts, 2 mM MgSO₄, 0.1 mM CaCl₂, 60 g/L glucose) media containing acetoacetate (10 mM) and/or butyrate (20 mM or 40 mM), followed by the HPLC analysis of the culture (Table 2). In Table 2, ‘10-M9-200-2-72h’ indicates the culturing in a M9 medium containing 10 mM acetoacetate and 20 mM butyrate for 72 hr, ‘400’ represents 40 mM butyrate, and represents a control cultured in a medium without butyrate.

As a result, as shown in Table 2, butanol was detected in the medium containing acetoacetate alone, as well as in the medium containing both acetoacetate and butyrate, regardless of the kind of medium.

TABLE 2
HPLC analysis of supernatants from cultures of E. coli ATCC 11303(pACT)
challenged with acetoacetate (10 mM) and/or butyric acid (20 mM or 40 mM)

All concentrations are given in mM

| Sample | Glucose | Acetate | Acetoin | Ethanol | Butyrate | Acetone | Butanol |
|---|---|---|---|---|---|---|---|
| 10-M9-C-2-24h | 401.5648 | 131.163 | | 67.34 | | 25.693 | |
| 10-M9-200-2-24h | 380.6775 | 287.421 | | 500.036 | | 5.722 | |
| 10-M9-400-2-24h | 156.5175 | 33.422 | | 17.776 | 39.739 | 7.661 | 0.476 |
| 10-LB-C-2-24h | 158.4865 | 17.207 | | 3.575 | | 1.794 | 0.492 |
| 10-LB-200-2-24h | 160.5093 | 16.512 | | 4.531 | | 2.061 | 0.503 |
| 10-LB-400-2-24h | 125.2173 | 16.382 | | 5.232 | | 2.941 | 0.59 |
| 10-M9-C-2-72h | 396.3895 | 148.93 | | 76.658 | | 29.12 | 0.548 |
| 10-M9-200-2-72h | 365.218 | 141.408 | | 49.579 | 22.871 | 35.363 | 0.746 |
| 10-M9-400-2-72h | 124.3028 | 17.975 | | 9.3 | | 3.628 | 0.899 |
| 10-LB-C-2-72h | 156.8248 | 19.419 | | 7.158 | | 2.794 | 0.812 |
| 10-LB-200-2-72h | 159.6945 | 19.127 | | 8.539 | | 3.151 | 0.89 |
| 10-LB-400-2-72h | 154.3995 | 34.977 | | 19.454 | 39.304 | 7.176 | 0.645 |
| 10-M9-C-2-96h | 286.3553 | 118.666 | | 59.421 | | 17.483 | 0.55 |
| 10-M9-200-2-96h | 271.9225 | 107.956 | | 34.066 | 25.704 | 8.192 | 0.772 |
| 10-M9-400-2-96h | 114.623 | 26.804 | | 16.284 | 50.433 | 5.754 | 0.67 |
| 10-LB-C-2-96h | 108.9373 | 19.842 | | 11.458 | | 2.342 | 0.782 |
| 10-LB-200-2-96h | 107.5158 | 17.616 | | 12.413 | 14.501 | 2.916 | 0.769 |
| 10-LB-400-2-96h | 94.365 | 14.925 | | 11.088 | | 3.086 | 0.927 |

### Example 3

### Production of Butanol in Wild-Type E. Coli by Addition of Acetoacetate or Butyrate

Wild-type E. coli (ATCC 11303) was pre-cultured for 24 hrs in 15 ml of a culture medium (LB containing 30 g/L glucose) in a culture tube. At an OD of 2.02, the culture was inoculated into a 500 ml medium in a flask. After being incubated to an OD of 0.4, the resulting culture was aliquoted into two 250 ml bottles. When the OD reached 0.42, the culture bottles were centrifuged at 5000 rpm for 10 min to discard the supernatant. The bottles were put in an aerobic chamber and added with 30 ml of a fresh medium in the anaerobic chamber, respectively. 300 μl of 0.108 g/ml lithium acetoacetate (Sigma, A-8509) solution was added to each tube to a final concentration of 10 mM, and butyrate was also added to a final concentration of 0.8 mM. The pH of the culture medium was adjusted to 6.25 which is that of the culture medium before the addition of butyrate. After cells were suspended and cultured, the final culture broth was analyzed using HPLC (Table 3).

In Table 3, ‘L8-200-72h’ indicates the culturing in an LB medium containing 10 mM acetoacetate and 0.8 mM butyrate for 72 hr, and ‘LC8’ indicates the culturing in a medium containing acetoacetate alone without butyrate, as a control.

As a result, as shown in Table 3, it was confirmed that wild-type E. coli (ATCC 11303) produces butanol when cultured in the medium containing both acetoacetate and butyrate, as it can be shown in Example 1. In addition, butanol was detected in the medium containing acetoacetate alone.

TABLE 3
HPLC analysis of supernatants from cultures of E. coli ATCC 11303
(w/o pACT) challenged with acetoacetate (10 mM) and/or butyric acid (0.8 mM)

All concentrations are given in mM

| Sample | Glucose | Acetate | Acetoin | Ethanol | Butyrate | Acetone | Butanol |
|---|---|---|---|---|---|---|---|
| LC8-24h | 160.4175 | 13.426 | | 4.585 | | 6.803 | |
| LC8-48h | 159.9535 | 12.81 | | 5.034 | | 5.842 | |
| LC8-72h | 159.5753 | 11.948 | | 5.943 | | 4.812 | |
| LC8-96h | 144.644 | 11.74 | | 6.575 | | 3.767 | |
| LC8-120h | 145.9135 | 11.399 | | 7.499 | | 3.167 | |
| LC8-144h | 158.6893 | 13.765 | | 9.988 | | 3.012 | 0.067 |
| LC8-168h | 148.1663 | 7.301 | | 12.385 | | 2.718 | 0.114 |
| LC8-192h | 149.9288 | 7.003 | | 12.764 | | 2.285 | 0.088 |
| LC8-264h | 155.7643 | 18.58 | | 15.3 | | 1.389 | 0.544 |
| LC8-288h | 155.3868 | 16.797 | | 17.72 | | 0.991 | 0.459 |
| L8-200-24h | 155.5293 | 12.876 | | 5.607 | 24.055 | 7.712 | |
| L8-200-48h | 155.6293 | 12.086 | | 4.834 | 21.089 | 6.045 | |
| L8-200-72h | 155.1658 | 12.701 | | 5.854 | 20.407 | 5.578 | |
| L8-200-96h | 141.744 | 12.934 | | 6.426 | 18.852 | 4.661 | 0.021 |
| L8-200-120h | 147.2203 | 13.557 | | 7.486 | 19.539 | 3.922 | 0.069 |
| L8-200-144h | 150.471 | 7.1 | | 10.369 | 19.975 | 4.047 | 0.093 |
| L8-200-192h | 147.4355 | 11.333 | | 14.416 | 19.954 | 2.574 | 0.147 |
| L8-200-264h | 151.9705 | 17.761 | | 16.967 | 22.17 | 1.107 | 0.543 |
| L8-200-288h | 150.526 | 16.271 | | 18.951 | 22.319 | 0.846 | 0.516 |

### Example 4

### Production of Butanol in E. Coli, into which a Pathway for Producing Butyryl-CoA from Acetyl-CoA is Introduced

4-1: Construction of pKKhbdthiL Vector

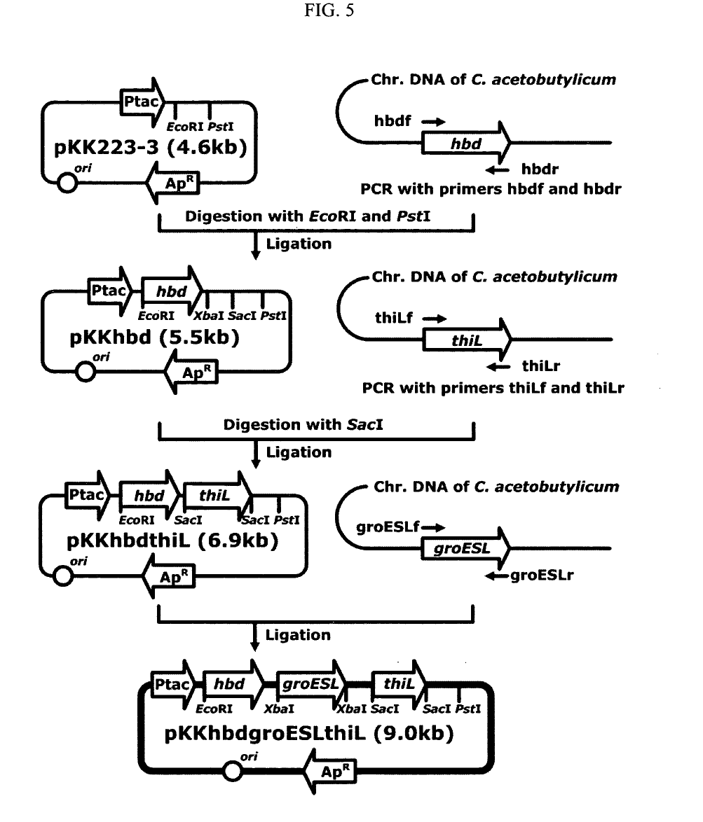

Genes necessary for the butanol biosynthesis pathway, including hbd (coding for 3-hydroxybutyryl-CoA dehydrogenase) and thiL (coding for thiolase) were amplified and sequentially cloned into a pKK223-3 expression vector (Pharmacia Biotech), thus constructed a recombinant expression vector, named pKKhbdthiL (FIG. 5).

PCR was performed on the chromosomal DNA of Clostridium acetobutylicum (KCTC 1724) using primers of SEQ ID NOS: 1 and 2, with 24 cycles of denaturing at 95° C. for 20 sec, annealing at 55° C. for 30 sec, and extending at 72° C. for 1 min. The PCR product (hbd gene) obtained was digested with EcoRI and PstI to clone into a pKK223-3 expression vector (Pharmacia Biotech), thus constructed a pKKhbd expression vector (FIG. 5).

To construct a pKKhbdthiL vector, PCR was first performed using primers of SEQ ID NOS: 3 and 4. The PCR product (a thiL gene) obtained was treated with SacI and then inserted into the pKKhbd vector digested with the same restriction enzyme (SacI), thus constructed a pKKhbdthiL vector containing both an hbd gene and a thiL gene (FIG. 5).

| [SEQ ID NO: 1] | |
|---|---|
| hbdf: | 5′-acgcgaattcatgaaaaaggtatgtgttat-3′ |
| [SEQ ID NO: 2] | |
| hbdr: | 5′-gcgtctgcaggagctcctgtctctagaatttgataatggggattctt-3′ |
| [SEQ ID NO: 3] | |
| thiLf: | 5′-acgcgagctctatagaattggtaaggatat-3′ |
| [SEQ ID NO: 4] | |
| thiLr: | 5′-gcgtgagctcattgaacctccttaataact-3′ |

To construct a pKKhbdgroESLthiL vector, PCR was performed using primers of SEQ ID NOS: 5 and 6, with the chromosomal DNA of Clostridium acetobutylicum serving as a template. The PCR product (a groESL gene) obtained was cleaved with XbaI and then inserted into the pKKhbdthiL digested with the same restriction enzyme (XbaI), thus constructed a pKKhbdgroESLthiL vector (FIG. 5).

[SEQ ID NO: 5]
groESLf: 5′-agcttctagactcaagattaacgagtgcta-3′
[SEQ ID NO: 6]
groESLr: 5′-tagctctagattagtacattccgcccattc-3′

4-2: Construction of pTrc184bcdcrt Vector

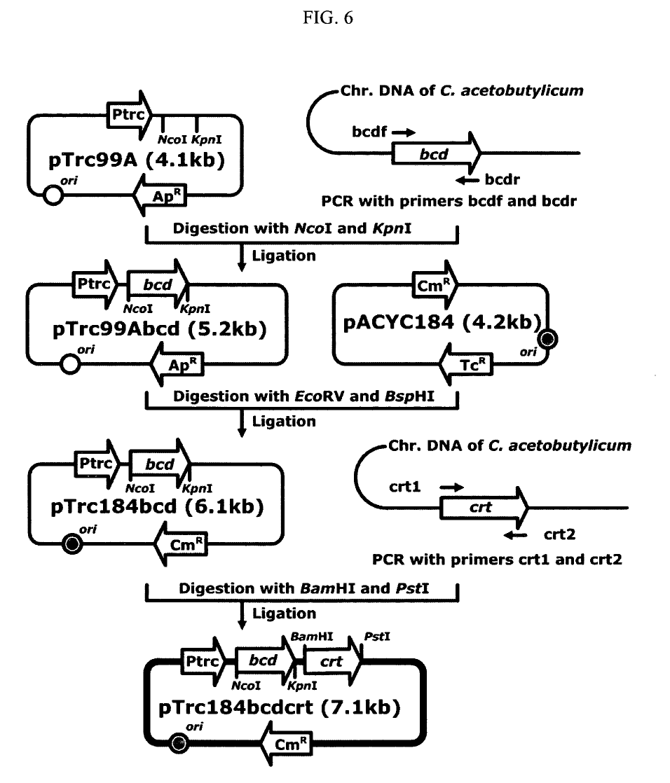

PCR was performed using primers of SEQ ID NOS: 7 and 8, with the chromosomal DNA of Clostridium acetobutylicum serving as a template. The PCR product (bcd gene) obtained was digested with NcoI and KpnI and cloned into a pTrc99A expression vector (Amersham Pharmacia Biotech), thus constructed a pTrc99Abcd vector. A DNA fragment excised from the pTrc99Abcd vector by digestion with BspHI and EcoRV was inserted into pACYC 184 (New England Biolabs) digested with the same restriction enzymes (BspHI and EcoRV), thus constructed a pTrc184bcd vector containing a bcd gene (FIG. 6).

[SEQ ID NO: 7]
bcdf: 5′-agcgccatggattttaatttaacaag-3′
[SEQ ID NO: 8]
bcdr: 5′-agtcggtacccctccttaaattatctaaaa-3′

To construct a pTrc184bcdcrt vector, PCR was performed using primers of SEQ ID NOS: 9 and 10. The PCR product (crt gene) obtained was digested with BamHI and PstI and then inserted into the pTrc184bcd digested with the same restriction enzymes (BamHI and PstI), thus constructed a pTrc184bcdcrt vector containing a bcd gene and a crt gene (FIG. 6).

[SEQ ID NO: 9]
crt1: 5′-atacggatccgagattagtacggtaatgtt-3′
[SEQ ID NO: 10]
crt2: 5′-gtacctgcagettacctcctatctattttt-3′

4-3: Deletion of lacI Gene

The lacI gene on the chromosomal DNA was deleted, so that a tac promoter and a trc promoter contained in the recombinant vectors prepared in Examples 4-1 and 4-2 could be operated constitutively, thus leading to the constitutive expression of the genes (hbd, thiL, groESL, bcd and crt) cloned into the corresponding vectors. In E. coli W3110 (ATTC 39936) containing a gene coding for an enzyme (AdhE) responsible for the conversion of butyryl-CoA to butanol, the lacI gene, which codes for the lac operon repressor and functions to inhibit the transcription of a lac operon required for the metabolism of lactose, was deleted through one-step inactivation (Warner et al., PNAS, 6:97(12):6640, 2000) using primers of SEQ ID NOS: 11 and 12, followed by the removal of antibiotic resistance from the bacterium, thus prepared a novel WL strain.

[SEQ ID NO: 11]
lacI_1stup:
5′-gtgaaaccagtaacgttatacgatgtcgcagagtatgccggtgtctc
ttagattgcagcattacacgtcttg-3′
[SEQ ID NO: 12]
lacI_1stdo:
5′-tcactgcccgctttccagtcgggaaacctgtcgtgccagctgcatta
atgcacttaacggctgacatggg-3′

### 4-4: Preparation of Butanol-Producing Microorganism

Both the pKKhbdgroESLthiL vector and the pTrc184bcdcrt vector prepared in Examples 4-1 and 4-2 were introduced into the WL strain of Example 4-3, thus prepared a novel butanol-producing recombinant microorganism (WL+pKKhbdgroESLthiL+pTrc184bcdcrt).

### 4-5: Assay for Butanol Productivity

The butanol-producing microorganism prepared in Example 4-4 was selected on LB plates containing 50 μg/ml ampicillin and 30 μg/ml chloramphenicol. The recombinants were precultured at 37° C. for 12 hrs in 10 ml of an LB medium. Then, after being autoclaved, 100 mL of LB medium maintained at 80° C. or higher in a 250 mL flask was added with glucose (10 g/L) and cooled to room temperature in an anaerobic chamber purged with nitrogen gas. 2 mL of the preculture broth was inoculated into the flask and cultured at 37° C. When the glucose of the medium was completely exhausted, as measured using a glucose analyzer (STAT, Yellow Springs Instrument, Yellow Springs, Ohio, USA), the broth was taken and analyzed for butanol concentration using gas chromatography (Agilent 6890N GC System, Agilent Technologies Inc., CA, USA) equipped with a packed column (Supelco Carbopack™ B AW/6.6% PEG 20M, 2 m×2 mm ID, Bellefonte, Pa., USA).

The results are summarized in Table 4, below. As seen in Table 4, wild-type E. coli W3110 did not produce butanol, whereas the recombinant mutant microorganism according to the present invention produced butanol. Taken together, the data obtained above demonstrate that butyryl-CoA is successfully produced from acetyl-CoA as a result of the overexpression of hbd, thiL, bcd, groESL and crt genes, and converted into butanol by the AdhE enzyme present per se in E. coli.

TABLE 4

| Strain | Butanol (mg/L) |
|---|---|
| W3110 | ND¹ |
| WL + pKKhbdgroESLthiL + pTrc184bcdcrt | 0.85 |

¹Not detected.

### Example 5

### Production of Butanol from Recombinant Microorganisms Introduced with Foreign Genes

5-1: Deletion of ldhA Gene

In the lacI-knockout E. coli W3110 of Example 4-3, ldhA (coding for lactate dehydrogenase) was further deleted by one-step inactivation using primers of SEQ ID NOS: 13 to 14. Thereby, WLL strain was prepared.

[SEQ ID NO: 13]
ldhA1stup:
5′-atgaaactcgccgtttatagcacaaaacagtacgacaagaagtacct
gcagattgcagcattacacgtcttg-3′
[SEQ ID NO: 14]
ldhA1stdo:
5′-ttaaaccagttcgttcgggcaggtttcgcctttttccagattgctta
agtcacttaacggctgacatggga-3′

5-2: Construction of pKKhbdadhEthiL (pKKHAT) Vector

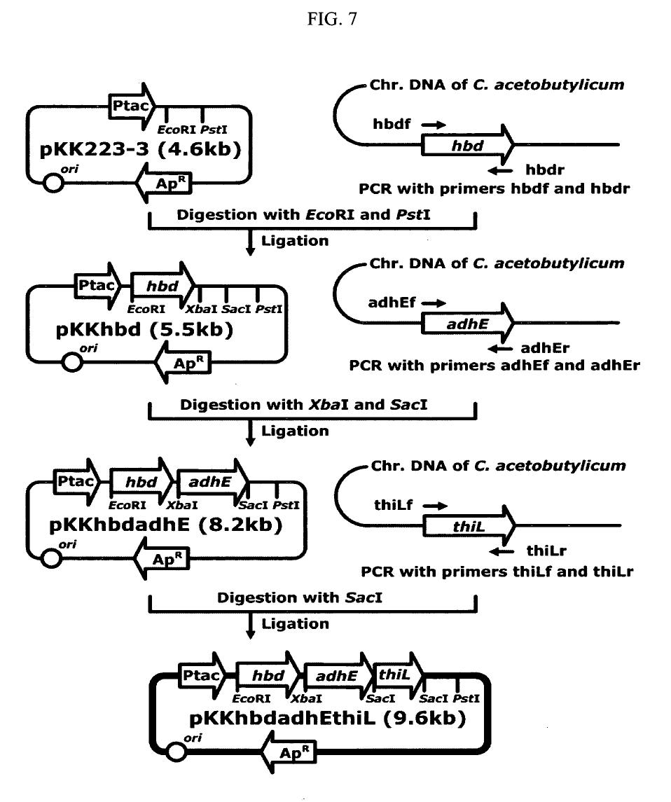

Genes necessary for the butanol biosynthesis pathway, including hbd (coding for 3-hydroxybutyryl-CoA dehydrogenase), adhE (coding for butyraldehyde dehydrogenase: the same spell, but different in function from the adhE (coding for alcohol dehydrogenase) of 1-2) and thiL (coding for thiolase) was amplified using primers of SEQ ID NOS: 15 to 20 with the chromosomal DNA of Clostridium acetobutylicum (KCTC 1724) serving as a template, and they were sequentially cloned into a pKK223-3 expression vector (Pharmacia Biotech), thus constructed a recombinant expression vector, named pKKhbdadhEthiL (pKKHAT) (FIG. 7).

| [SEQ ID NO: 15] | |
|---|---|
| hbdf: | 5′-acgcgaattcatgaaaaaggtatgtgttat-3′ |
| [SEQ ID NO: 16] | |
| hbdr: | 5′-gcgtctgcaggagctcctgtctctagaatttgataatggggattctt-3′ |
| [SEQ ID NO: 17] | |
| adhEf: | 5′-acgctctagatataaggcatcaaagtgtgt-3′ |
| [SEQ ID NO: 18] | |
| adhEr: | 5′-gcgtgagctccatgaagctaatataatgaa-3′ |
| [SEQ ID NO: 19] | |
| thiLf: | 5′-acgcgagctctatagaattggtaaggatat-3′ |
| [SEQ ID NO: 20] | |
| thiLr: | 5′-gcgtgagctcattgaacctccttaataact-3′ |

5-3: Construction of pKKhbdadhEatoB (pKKHAA) Vector

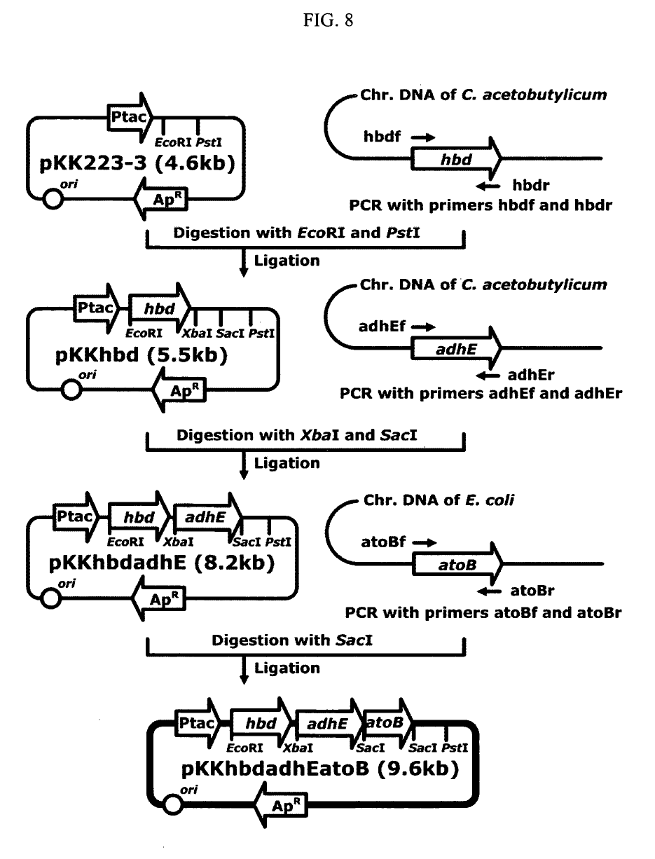

To clone the atoB (coding for acetyl-CoA acetyltransferase) of Escherichia coli W3110 into the pKKhbdadhE vector (FIG. 7), PCR was performed on the chromosomal DNA of Escherichia coli W3110 using primers of SEQ ID NOS: 21 and 22, with 24 cycles of denaturing at 95° C. for 20 sec, annealing at 55° C. for 30 sec and extending at 72° C. for 90 sec. The PCR product (atoB) obtained was digested with SacI and inserted into the pKKhbdadhE vector digested with the same restriction enzyme (SacI), thus constructed a novel recombinant vector, named pKKhbdadhEatoB (pKKHAA) (FIG. 8).

[SEQ ID NO: 21]
atof: 5′-atacgagctctacggcgagcaatggatgaa-3′
[SEQ ID NO: 22]
ator: 5′-gtacgagctcgattaattcaaccgttcaat-3′

5-4: Construction of pKKhbdadhEphaA (pKKHAP) Vector

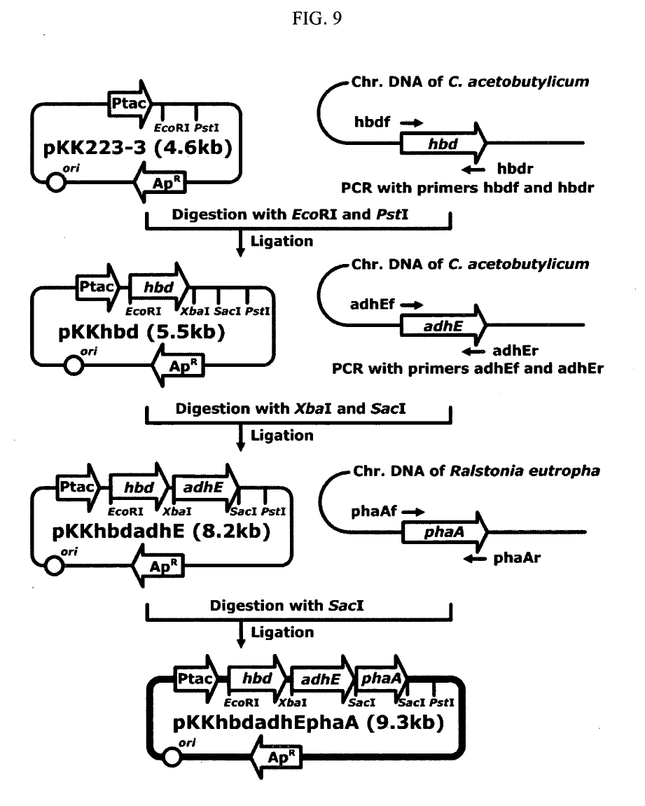

To clone the phaA (coding for thiolase) of Ralstonia eutropha (KCTC 1006) into the pKKhbdadhE vector, PCR was performed using primers of SEQ ID NOS: 23 and 24, with the chromosomal DNA of Ralstonia eutropha serving as a template. The PCR product (phaA) obtained was cleaved with SacI and inserted into the pKKhbdadhE vector digested with the same restriction enzyme (SacI), thus constructed a novel recombinant vector, named pKKhbdadhEphaA (pKKHAP) (FIG. 9).

[SEQ ID NO: 23]
phaAf:
5′-agtcgagctcaggaaacagatgactgacgttgtcatcgt-3′
[SEQ ID NO: 24]
phaAr:
5′-atgcgagctcttatttgcgctcgactgcca-3′

5-5: Construction of pKKhbdydbMadhEphaA (pKKHYAP) Vector

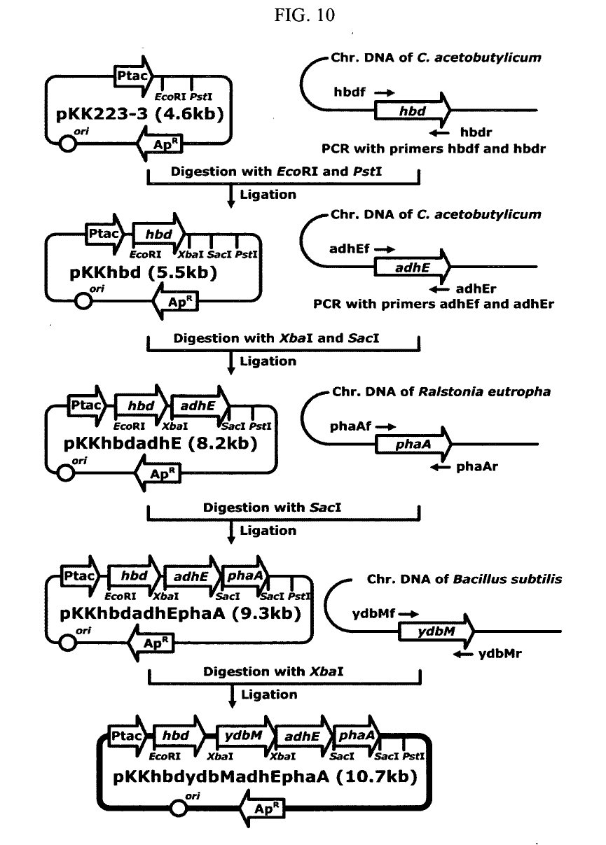

To clone the ydbM (coding for hypothetical protein) of Bacillus subtilis (KCTC 1022) into the pKKhbdadhE vector, PCR was performed using primers of SEQ ID NOS: 25 and 26 with the chromosomal DNA of Bacillus subtilis serving as a template. The PCR product (ydbM) obtained was cleaved with XbaI and inserted into the pKKhbdadhEphaA vector digested with the same restriction enzyme (XbaI), thus constructed a novel recombinant vector, named pKKhbdydbMadhEphaA (pKKHYAP) (FIG. 10).

[SEQ ID NO: 25]
ydbMf: 5′-agcttctagagatgggttacctgacatata-3′
[SEQ ID NO: 26]
ydbMr: 5′-agtctctagattatgactcaaacgcttcag-3′

5-6: Construction of pKKhbdbcdPA01adhEphaA (pKKHPAP) Vector

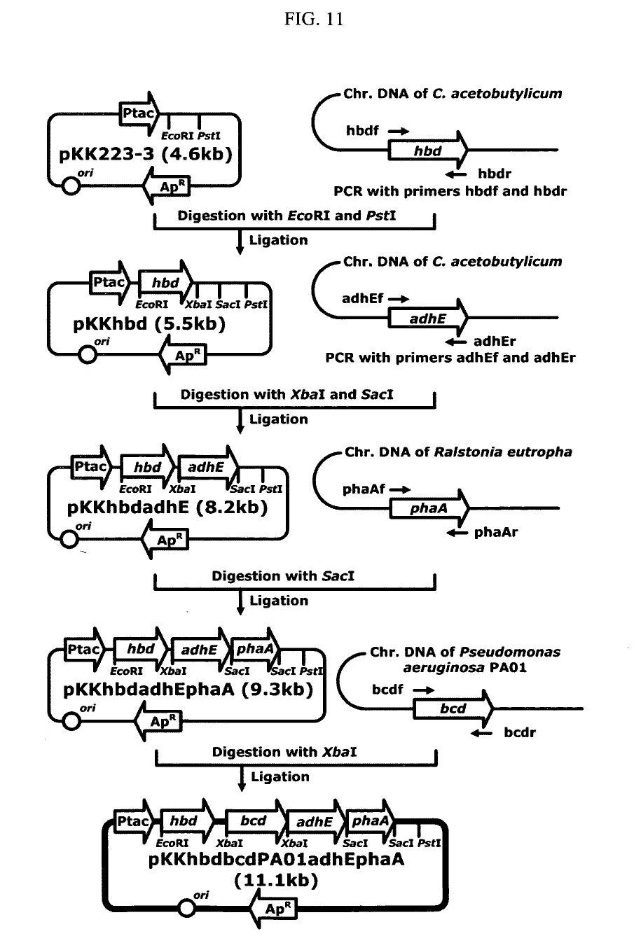

To clone the bcd (coding for butyryl-CoA dehydrogenase) of Pseudomonas aeruginosa PA01 (KCTC 1637) into the pKKhbdadhEphaA vector, PCR was performed using primers of SEQ ID NOS: 27 and 28 with the chromosomal DNA of Pseudomonas aeruginosa PA01 serving as a template. The PCR product (bcd) obtained was cleaved with XbaI and inserted into the pKKhbdadhEphaA (pKKHAP) vector digested with the same restriction enzyme (XbaI), thus constructed a novel recombinant vector, named pKKhbdbcdPA01adhEphaA (pKKHPAP) (FIG. 11).

[SEQ ID NO: 27]
bcdPA01f: 5′-agcttctagaactgctccttggacagcgcc-3′
[SEQ ID NO: 28]
bcdPA01r: 5′-agtctctagaggcaggcaggatcagaacca-3′

5-7: Construction of pKKhbdbcdKT2440adhEphaA (pKKHKAP) Vector

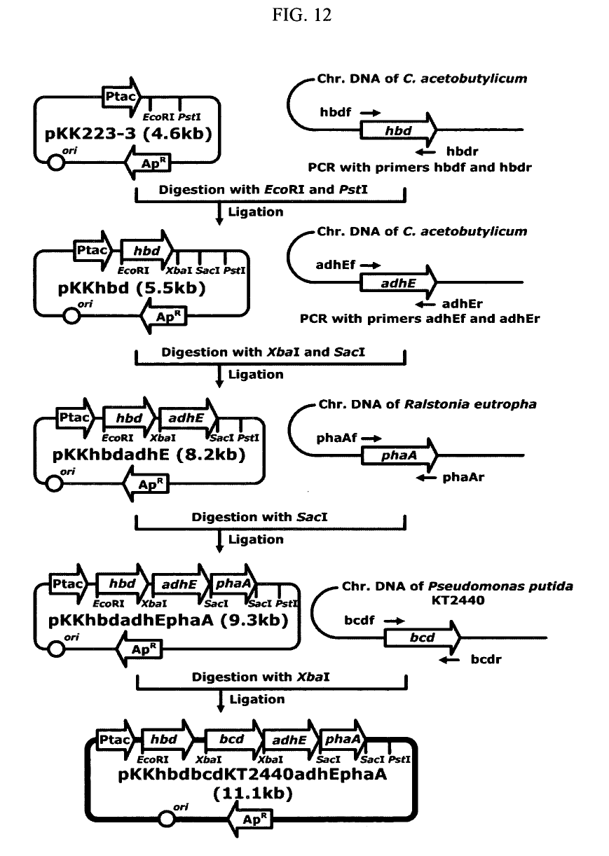

To clone the bcd (coding for butyryl-CoA dehydrogenase) of Pseudomonas putida KT2440 (KCTC 1134) into the pKKhbdadhEphaA vector, PCR was performed using primers of SEQ ID NOS: 29 and 30 with the chromosomal DNA of Pseudomonas putida KT2440 serving as a template. The PCR product (bcd) obtained was cleaved with XbaI and inserted into the pKKhbdadhEphaA vector digested with the same restriction enzyme (XbaI), thus constructed a novel recombinant vector, named pKKhbdbcdKT2440adhEphaA (pKKHKAP) (FIG. 12).

[SEQ ID NO: 29]
bcdKT2440f: 5′-agcttctagaactgttccttggacagcgcc-3′
[SEQ ID NO: 30]
bcdKT2440r: 5′-agtactagaggcaggcaggatcagaacca-3′

5-8: Construction of pTrc184bcdbdhABcrt Vector

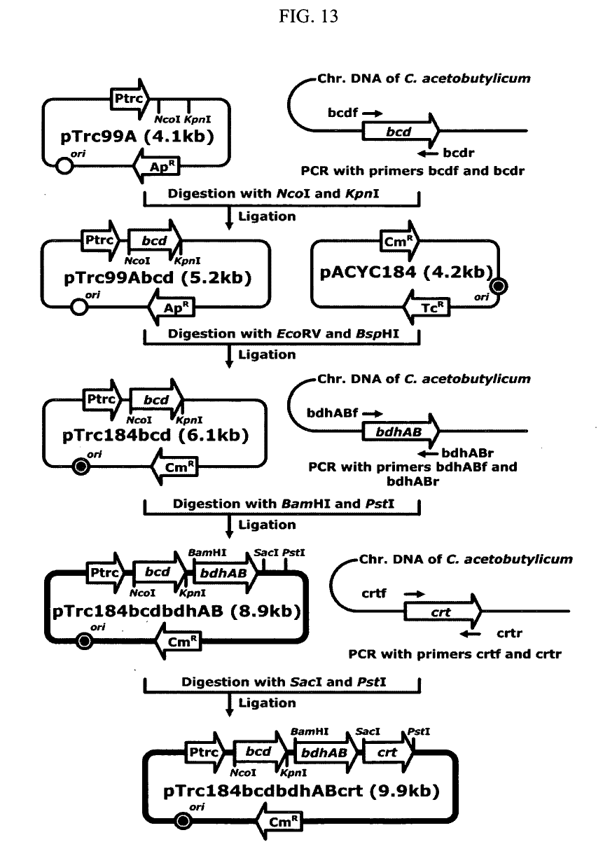

PCR was performed using primers of SEQ ID NOS: 31 and 32, with the chromosomal DNA of Clostridium acetobutylicum serving as a template. The PCR product (bcd) obtained was digested with NcoI and KpnI and cloned into a pTrc99A expression vector (Amersham Pharmacia Biotech), thus constructed a recombinant vector named pTrc99Abcd. After the pTrc99Abcd vector was digested with BspHI and EcoRV, the DNA fragment thus excised was inserted into pACYC184 (New England Biolabs) which was previously treated with the same restriction enzymes (BspHI and EcoRV), thus constructed a recombinant expression vector for expressing the bcd gene, named pTrc184bcd (FIG. 13).

[SEQ ID NO: 31]
bcdf: 5′-agcgccatggattttaatttaacaag-3′
[SEQ ID NO: 32]
bcdr: 5′-agteggtacccctecttaaattatctaaaa-3′

PCR was performed using primers of SEQ ID NOS: 33 and 34, with the chromosomal DNA of Clostridium acetobutylicum serving as a template. The PCR product (bdhAB) obtained was digested with BamHI and PstI and inserted into the pTrc184bcd expression vector digested with the same restriction enzymes (BamHI and PstI), thus constructed a recombinant vector, named pTrc184bcdbdhAB (pTrc184BB), which contained both bcd and bdhAB.

[SEQ ID NO: 33]
bdhABf:
5′-acgcggatccgtagtttgcatgaaatttcg-3′
[SEQ ID NO: 34]
bdhABr:
5′-agtcctgcagctatcgagctctataatggctacgcccaaac-3′

PCR was performed using primers of SEQ ID NOS: 35 and 36, with the chromosomal DNA of Clostridium acetobutylicum serving as a template. The PCR product (crt) obtained was digested with SacI and PstI and inserted into the pTrc184bcdbdhAB expression vector digested with the same restriction enzymes (SacI and PstI), thus constructed a recombinant vector, named pTrc184bcdbdhABcrt (pTrc184BBC), which contained all of the bcd gene, the bdhAB gene and the crt gene (FIG. 13).

[SEQ ID NO: 35]
crtf: 5′-actcgagctcaaaagccgagattagtacgg-3′
[SEQ ID NO: 36]
crtr: 5′-gcgtctgcagcctatctatttttgaagcct-3′

### 5-9: Preparation of Butanol-Producing Microorganisms

E. coli W3110 (WLL) lacking lacI and ldhA prepared in Examples 5-1, was transformed with the pTrc184bcdbdhABcrt (pTrc184BBC) vector of Example 5-8 and the vector selected from the group consisting of pKKhbdadhEthiL (pKKHAT), pKKhbdadhEatoB (pKKHAA), pKKhbdydbMadhEphaA (pKKHYAP), pKKhbdadhEphaA (pKKHAP), pKKhbdbcdPA01adhEphaA (pKKHPAP), and pKKhbdbcdKT2440adhEphaA (pKKHKAP) constructed in Examples 5-2 to 5-7, thus prepared recombinant mutant microorganisms (WLL+pKKHAT+pTrc184BBC, WLL+pKKHAA+pTrc184BBC, WLL+pKKHAP+pTrc184BBC, WLL+pKKHYAP+pTrc184BBC, WLL+pKKHPAP+pTrc184BBC, and WLL+pKKHKAP+pTrc184BBC) capable of producing butanol.

### 5-10: Assay for Butanol Productivity

The butanol-producing microorganisms prepared in Example 5-9 were selected on LB plates containing 50 μg/ml ampicillin and 30 μg/ml chloramphenicol. For the selection of the WLLPA+pKKHPAP+pTrc184BBC strain, kanamycin was added in an amount of 30 μg/ml to the LB plates. The recombinants were precultured at 37° C. for 12 hr in 10 ml of LB broth. After being autoclaved, 100 mL of LB broth maintained at 80° C. or higher in a 250 mL flask was added with glucose (5 g/L) and cooled to room temperature in an anaerobic chamber purged with nitrogen gas. 2 mL of the preculture was inoculated into the flask and cultured at 37° C. for 10 hr. Then, 2.0 liters of a medium containing 20 g of glucose, 2 g of KH₂PO₄, 15 g of (NH₄)₂SO₄.7H₂O, 20 mg of MnSO₄.5H₂O, 2 g of MgSO₄.7H₂O, 3 g of yeast extract, and 5 ml of a trace metal solution (10 g FeSO₄.7H₂O, 1.35 g CaCl₂, 2.25 g ZnSO₄.7H₂O, 0.5 g MnSO₄.4H₂O, 1 g CuSO₄.5H₂O, 0.106 g (NH₄)₆Mo₇O₂₄.4H₂O, 0.23 g Na₂B₄O₇.10H₂O, and 35% HCl 10 ml per liter of distilled water) per liter of distilled water in a 5 L fermenter (LiFlus GX, Biotron Inc., Korea) was autoclaved and cooled from 80° C. or higher to room temperature with nitrogen supplied at a rate of 0.5 vvm for 10 hr. In the fermenter, the culture was carried out at 37° C., 200 rpm with shaking at 200 rpm. During the cultutivation, pH to be maintained at 6.8 by automatic feeding with 25% (v/v) NH₄OH and nitrogen gas was supplied at a rate of 0.2 vvm (air volume/working volume/minute).

When the glucose of the medium was completely exhausted, as measured using a glucose analyzer (STAT, Yellow Springs Instrument, Yellow Springs, Ohio, USA), the medium was analyzed for butanol concentration using gas chromatography (Agillent 6890N GC System, Agilent Technologies Inc., CA, USA) equipped with a packed column (Supelco Carbopack™ B AW/6.6% PEG 20M, 2 m×2 mm ID, Bellefonte, Pa., USA).

As a result, as shown in Table 5, butanol was produced by the cells, into which thiL (WLL+pKKHAT+pTrc184BBC), phaA (WLL+pKKHAP+pTrc184BBC) or atoB (WLL+pKKHAA+pTrc184BBC) as a gene encoding THL was introduced. From this result, it could be confirmed that exogenous gene encoding THL can also be expressed to show THL activity in host cells such E. coli.

Also, the butanol production data show that, compared to the case where only the bcd derived from Clostridium acetobutylicum was introduced (WLL+pKKHAP+pTrc184BBC), butyryl-CoA dehydrogenase activity increased in the case where the bcd derived from Clostridium acetobutylicum was introduced together with the ydbM derived from Bacillus subtilis (WLL+pKKHYAP+pTrc184BBC) or with the bcd derived from Pseudomonas aeruginosa or Pseudomonas putida (WLL+pKKHPAP+pTrc184BBC; WLL+pKKHKAP+pTrc184BBC). From this result, it could be confirmed that exogenous genes coding for BCD can also be expressed to show butyryl-CoA dehydrogenase activity in host cells such as E. coli.

TABLE 5

| | | Butanol |
|---|---|---|
| Strains | Containing genes | (mg/L) |
| W3110 | — | ND¹ |
| WLL + pKKHAT + | hbd, adhE, thiL, bcd, bdhAB, crt | 1.2 |
| pTrc184BBC | | |
| WLL + pKKHAA + | hbd, adhE, atoB, bcd, bdhAB, crt | 1.3 |
| pTrc184BBC | | |
| WLL + pKKHAP + | hbd, adhE, phaA, bcd, bdhAB, crt | 1.4 |
| pTrc184BBC | | |
| WLL + pKKHYAP + | hbd, adhE, ydbM, phaA, bcd, bdhAB, | 1.7 |
| pTrc184BBC | crt | |
| WLL + pKKHPAP + | hbd, adhE, bcdPA01, phaA, bcd, | 3.1 |
| pTrc184BBC | bdhAB, crt | |
| WLL + pKKHKAP + | hbd, adhE, bcdKT2440, phaA, bcd, | 9.1 |
| pTrc184BBC | bdhAB, crt | |

¹Not detected.

### Example 6

### Production of Butanol from Recombinant Microorganisms Introduced with Genes Derived from E. Coli and C. Acetobutylicum

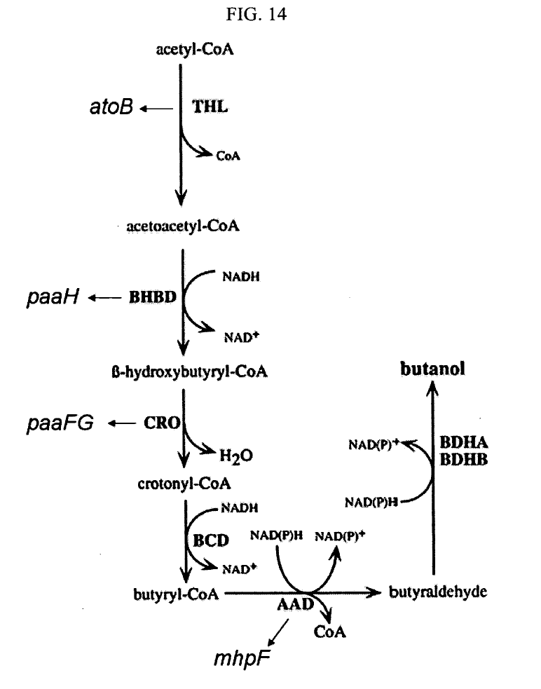

In this example, when the genes derived from C. acetobutylicum, responsible for the butanol biosynthesis pathway, were partially substituted with genes derived from E. coli, butanol productivity was measured (FIG. 14). It is well known that mhpF derived from E. coli encodes acetaldehyde dehydrogenase (Ferrandez, A. et al., J. Bacteriol., 179:2573, 1997). In this example, when adhE, crt, hbd and thiL, derived from Clostridium sp., were substituted with genes [mhpF (acetaldehyde dehydrogenase encoding gene), paaFG, paaH and atoB] derived from E. coli, respectively, the resulting recombinant microorganisms were measured for butanol productivity.

6-1: Construction of pKKmhpFpaaFGHatoB Vector

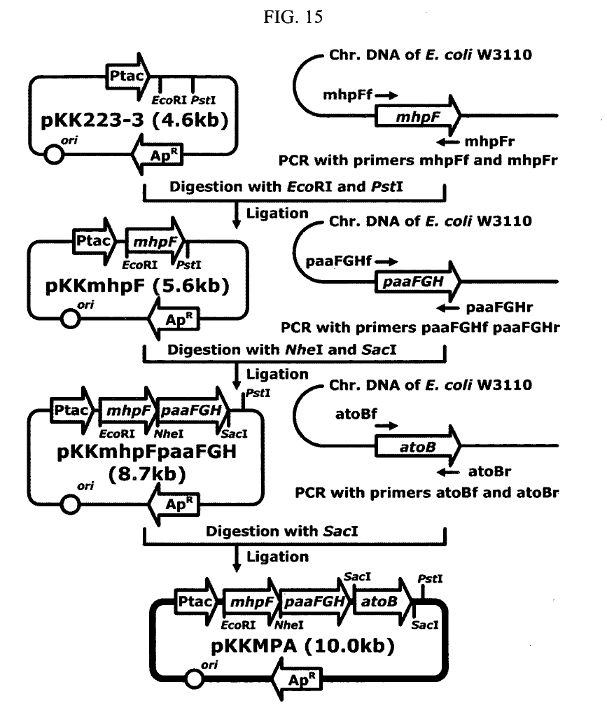

PCR was performed using primers of SEQ ID NOS: 37 to 42, with the chromosomal DNA of E. coli W3110 serving as a template, to amplify genes essential for the butanol biosynthesis pathway, including mhpF (coding for acetaldehyde dehydrogenase), paaFG (coding for enoyl-CoA hydratase), paaH (coding for 3-hydroxy-acyl-CoA dehydrogenase) and atoB (coding for acetyl-CoA acetyltransferase). These genes were sequentially cloned into a pKK223-3 expression vector (Pharmacia Biotech), thus constructed a novel recombinant expression vector, named pKKmhpFpaaFGHatoB (pKKMPA) (FIG. 15).

| [SEQ ID NO: 37] | |
|---|---|
| mhpFf: | 5′-atgcgaattcatgagtaagcgtaaagtcgc-3′ |
| [SEQ ID NO: 38] | |
| mhpFr: | 5′-tatcctgcaggagctctctagagctagcttaccgttcatgccgcttct-3′ |
| [SEQ ID NO: 39] | |
| paaFGHf: | 5′-atacgctagcatgaactggccgcaggttat-3′ |
| [SEQ ID NO: 40] | |
| paaFGHr: | 5′-tatcgagctcgccaggccttatgactcata-3′ |
| [SEQ ID NO: 41] | |
| atoBf: | 5′-atacgagctetgcatcactgccctgctctt-3′ |
| [SEQ ID NO: 42] | |
| atoBr: | 5′-tgtcgagctccgctatcgggtgtttttatt-3′ |

### 6-2: Preparation of Butanol-Producing Microorganism

E. coli W3110 (WLL) lacking lacI and ldhA, prepared in Example 5-1, was transformed with the pKKMPA vector of Example 6-1 and the pTrc184bcdbdhAB (pTrc184BB) vector of Example 5-8, thus prepared recombinant mutant microorganism capable of producing butanol (WLL+pKKMPA+pTrc184BB).

### 6-3 Assay for Butanol Productivity

The butanol-producing microorganism prepared in Example 6-2 was cultured in the same manner as in Example 5-10 and measured for butanol productivity under the same conditions.

As a result, as shown in Table 6, compared to when only the butanol biosynthesis pathway of C. acetobutylicum was used, butanol productivity was improved when E. coli-derived genes predicted to code the corresponding enzymes (adhE→mhpF, crt→paaFG, hbd→paaH, thiL→atoB) and the bcd and bdhAB genes derived from C. acetobutylicum were used in combination. That is, four (butyraldehyde dehydrogenase, crotonase, BHBD and THL) of the enzymes from Clostridium acetobutylicum essential for butanol production in E. coli can be substituted with enzymes encoded by mhpF, paaFG, paaH and atoB genes derived from E. coli, and these enzymes from E. coli were found to have higher activity than the corresponding enzymes from C. acetobutylicum, as demonstrated by the enhanced butanol production.

TABLE 6

| Strains | Containing genes | Butanol (mg/L) |
|---|---|---|
| WLL + pKKMPA + | mhpF, paaFGH, atoB, | 18.4 |
| pTrc184BB | bcd, bdhAB | |

### INDUSTRIAL APPLICABILITY

As described above in detail, the present invention has an effect to provide a method for producing butanol, which comprising generating butyryl-CoA in various ways and producing butanol using butyryl-CoA as an intermediate.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A method for producing butanol, the method comprising: culturing a recombinant bacterium containing a gene coding for an enzyme (AdhE) responsible for the conversion of butyryl-CoA to butanol, and into which a gene coding for CoAT (acetyl-CoA: butyryl-CoA transferase) is introduced in a butyrate or acetoacetate-containing culture medium to produce butanol; and recovering butanol from the culture broth.

2. The method for producing butanol according to claim 1, wherein a gene coding for thiolase (THL) and a gene coding acetoacetate decarboxylase (AADC) are additionally introduced into the recombinant bacterium.

3. The method for producing butanol according to claim 1, wherein said bacterium is E. coli.

4. The method for producing butanol according to claim 1, wherein the gene coding for CoAT (acetyl-CoA: butyryl-CoA transferase) is ctfA and ctfB.

5. The method for producing butanol according to claim 4, wherein said ctfA and ctfB are derived from Clostridium sp.

6. The method for producing butanol according to claim 2, wherein the gene coding for THL is thl or thiL derived from Clostridium sp., phaA derived from Ralstonia sp., or atoB derived from E. coli.

7. The method for producing butanol according to claim 2, wherein, the gene coding for AADC is adc derived from Clostridium sp.

8. The method for producing butanol according to claim 1, wherein said culturing is carried out in an anaerobic condition.

9.-13. (canceled)

14. A method for producing butanol, the method comprising: culturing a bacterium containing a gene coding for AtoDA and a gene coding for AdhE in a butyrate-containing culture medium to produce butanol; and recovering butanol from the culture broth.

15. The method for producing butanol according to claim 14, wherein said bacterium is E. coli.

16. (canceled)

17. (canceled)

18. A method for producing butanol, the method comprising: culturing a bacterium containing genes coding for AtoDA, FadB or PaaH, PaaFG and FadE together with a gene coding for AdhE in a butyrate- or acetoacetate-containing culture medium to produce butanol; and recovering butanol from the culture broth.

19. The method for producing butanol according to claim 18, wherein said bacterium is E. coli.

20.-37. (canceled)
